SEQUENCE LISTING (6 sequences)

SEQ ID NO: 1 - DNA, 20 bp, Artificial Sequence
  Synthetic intron 5 primer
gagccacgtgttgaagtcct

SEQ ID NO: 2 - DNA, 20 bp, Artificial Sequence
  Synthetic exon 6 primer
tttgtaaaaggctgcccggc

SEQ ID NO: 3 - DNA, 22 bp, Artificial Sequence
  Synthetic exon 19 forward primer
cacagccccagtgtccctcacc

SEQ ID NO: 4 - DNA, 24 bp, Artificial Sequence
  Synthetic exon 19 reverse primer
ggatgtggagatgagcagggtcta

SEQ ID NO: 5 - DNA, 22 bp, Artificial Sequence
  Synthetic exon 21 forward primer
tggcatgaacatgaccctgaat

SEQ ID NO: 6 - DNA, 22 bp, Artificial Sequence
  Synthetic exon 21 reverse primer
gcatcctcccctgcatgtgtta

(19) United States
(12) Patent Application Publication
(10) Pub. No.: US 2013/0017540 A1
(43) Pub. Date: Jan. 17, 2013

(54) IDENTIFICATION OF MUTATION TYPES ASSOCIATED WITH ACQUIRED RESISTANCE AND METHODS FOR USING SAME

(71) Applicants: Yun Yen, Arcadia, CA (US); Quiang Liu, Upland, CA (US)

(72) Inventors: Yun Yen, Arcadia, CA (US); Quiang Liu, Upland, CA (US)

(21) Appl. No.: 13/491,486

(22) Filed: Jun. 7, 2012

Related U.S. Application Data
(60) Provisional application No. 61/494,366, filed on Jun. 7, 2011.

(51) Int. Cl. C12Q 1/68; G06F 19/00

(52) U.S. Cl. 435/6.11; 435/6.12; 702/19

(57) ABSTRACT

Methods for identifying or classifying a gene mutation type associated with acquired drug resistance of cancer is provided. Said methods may include determining a total copy number (N) of a susceptible gene in a cancer cell, identifying a mutant copy number of the susceptible gene, determining a mutant copy number sufficient to cause acquired drug resistance (M); and comparing N with M to identify or classify the mutation type in the cancer cell.

### PRIORITY CLAIM

This application claims priority to U.S. Provisional Application No. 61/494,366, filed Jun. 7, 2011, the subject matter of which is hereby incorporated by reference as if fully set forth herein.

### BACKGROUND

There are several well-documented instances of cancers that can develop acquired resistance to a targeted therapeutic (e.g., kinase inhibitors) which are initially successful in the treatment of cancer. For example, targeted therapeutics such as the BCR-ABL tyrosine kinase inhibitor imatinib for chronic myeloid leukemia (CML) are widely used, but acquired drug resistance limits their broader success. Acquired drug resistance is a phenomenon in which after a drug is given, cancer cells still accumulate over time because of acquired mutations. Mutations can also be acquired before therapy, consistent with over-dispersed surviving bacteria colony numbers.

CML arises when an oncogenic BCR-ABL fusion gene occurs in a primitive hematopoietic stem cell, as may result after marrow exposures to ionizing radiation. Imatinib, a BCR-ABL tyrosine kinase inhibitor, leads to complete cytogenetic responses and infrequent relapses in most chronic phase CML patients. However, imatinib is inefficacious in patients in advanced phases. Many BCR-ABL mutations, such as a T315I mutation in the BCR-ABL kinase domain, have been identified as imatinib resistant in relapsed CML patients.

Similarly, erlotinib and gefitinib initially elicit significant responses in non-small-cell lung cancer (NSCLC) patients through inhibition of somatic mutation-activated EGFR, a tyrosine kinase, in cancer cells. However, many patients acquire resistance due to a secondary point mutation, such as T790M, in the mutation-activated EGFR gene.

Because acquired drug resistance affects different types of cancer, methods for determining the types or mutations, mechanisms or phenomena behind such resistance are desired to develop markers that may serve to determine susceptibility and to guide therapy.

### SUMMARY

In one embodiment, a method for identifying or classifying a gene mutation type associated with acquired drug resistance of cancer is provided. Said method may include determining a total copy number (N) of a susceptible gene in a cancer cell, identifying a mutant copy number of the susceptible gene, determining a mutant copy number sufficient to cause acquired drug resistance (M); and comparing N with M to identify or classify the mutation type in the cancer cell.

In some aspects, the mutation type is a single copy mutation type when N is 1 and M is 1 (M=N=1). In another aspect, the mutation type is a dominant mutation type when N is 2 or more and the mutant copy number sufficient to cause resistance is M=1. In another aspect, the mutation type is an intermediate mutation type when N is 3 or more and the mutant number sufficient to cause resistance is more than one, but less than the total copy number N (1<M<N). In another aspect, the mutation type is a recessive mutation type when N is 2 or more (N≧2) and the mutant copy number sufficient to cause resistance is equal to the number of gene copies (M=N2).

In another embodiment, a method for identifying a dominant mutation type associated with acquired drug resistance in a cancer cell or a population of cancer cells is provided. Such a method may include, for example, calculating a number of cells having 0, 1 or 2 mutant copies of a gene susceptible to acquired resistance at a series of predetermined time intervals, generating a series of simulated growth kinetics graphs for cells having 0, 1 or 2 mutant copies of the susceptible gene at each time interval, comparing the series of simulated growth kinetics graphs to experimentally determined growth kinetics data, and determining the cancer cells acquire resistance to a drug through a dominant mutation type when the series of simulated growth kinetics graphs fit the experimentally determined growth kinetics data.

In some aspects, the number of cells having 0, 1 or 2 mutant copies of the gene susceptible to acquired resistance is calculated based on computational models using an experimentally determined constant growth rate and an experimentally determined constant mutation rate. In some aspects, the experimentally determined constant growth rate is determined by counting cells on a hemocytometer at given time points after treatment with a drug associated with acquired resistance and the experimentally determined constant mutation rate may be determined by a soft agar colony formation assay. In some aspects, the experimental growth kinetics are determined by a cell viability assay after treatment with a drug associated with acquired resistance.

In another aspect, a method for selecting, modifying, monitoring or predicting a response to a cancer treatment regimen for a cancer patient is provided, wherein the method may include identifying a cancer cell as having one or more mutant copies associated with acquired resistance to one or more cancer drugs, determining a mutation type for each of the one or more mutant copies as in claim 1 and selecting, modifying or monitoring a cancer treatment regimen based on the mutation type.

In some aspects, the cancer drug, treatment or treatment regimen that is associated with acquired resistance according to the embodiments described herein may include, but is not limited to, imatinib, erlotinib or gefitinib.

### BRIEF DESCRIPTION OF THE DRAWINGS

FIG. 1 illustrates three types of genetic mutations associated with drug resistance according to some embodiments. Mutation type (A) shows a single copy mutation type, wherein each sensitive and resistant cell contains only a single copy of a susceptible gene, such as the BCR-ABL fusion gene. Two distinct types of tumor cells exist: sensitive (squared) and resistant (circled). The circular arrow represents the net growth which values may be positive (solid line) or negative (dotted line). Each pair of curved lines represents one copy of the susceptible gene and a cross represents one copy of a mutant gene. The straight arrow indicates occurrence of mutations. The mutation event may accompany a conversion from a sensitive cell into a resistant cell. Mutation type (B) shows a dominant mutation type, wherein each sensitive or resistant cell contains two copies of a susceptible gene. One mutant copy is sufficient for resistance. Mechanism (C) shows a recessive mutation type, wherein each sensitive or resistant cell contains two copies of a susceptible gene. Two mutant copies are required for resistance.

FIG. 2 shows a Sanger sequencing analysis of genomic DNA from individual resistant clonal cells. To determine whether KCL-22M cells contain either one or two copies of the T315I mutation per cell, genomic DNA pooled templates from individual KCL-22M cell clones were sequenced. In the example shown with downstream primer, the mutated T peak is 50% as high as that of the wild type C peak, suggesting that only one copy of the fusion BCR-ABL gene contain the T315I mutation per cell.

FIG. 3A shows observed cell growth kinetics with imatinib. X-axis represents time with units in days. Y-axis shows the number of viable cells averaged in triplicate with standard deviation. In the earlier days of the experiment, virtually all the cells were KCL-22 cells. When KCL-22 cells were treated with 2.5, 5 or 10 μM imatinib, cells underwent initial apoptosis but relapsed after eight to nine days, due to the emergence of the resistant KCL-22M cells. FIG. 3A is adapted from Yuan et al. 2010.

FIG. 3B illustrates simulated cell growth kinetics based on the dominant gene mutation type (mutation type B). At 2.5 μM imatinib, the number of initial KCL-22 sensitive cells having zero mutant copies per cell, S₀, was 500,000 and its growth rate, kₛ, were −0.245. The numbers of initial KCL-22M resistant cells having 1 or 2 mutant copies per cell, δ₀ and R₀, were zero and the growth rates, k_{δ} and kᵣ, were 0.730. Mutation or transfer rates, μ₀₁, μ₀₂, μ₁₂ as well as μ₀₀ and μ₁₁ are detailed in Table 4. At given days, the numbers of sensitive and resistant cells having 0, 1 and 2 mutant copies per cell, S(t), δ(t) and R(t), were calculated according to Eq 1 (below). Similar patterns were obtained when 120 resistant cells were assumed to pre-exist (r₀=0, δ₀=120). The effect of treatment with 2.5 μM imatinib in all cells having 0, 1 and 2 mutant copies of the point mutation is represented by P(t), which is the sum of S(t), δ(t) and R(t).

FIG. 3C. Simulated cell growth kinetics based on recessive gene mutation type C. At 2.5 μM imatinib, the number of initial sensitive cells having zero and 1 mutant copy per cell, S₀ and δ₀, was 500,000 and zero, respectively. Their growth rates, kₛ and k_{δ}, were −0.245. The numbers of initial KCL-22M resistant cells having 2 mutant copies per cell, R₀, was zero and its growth rate, kᵣ, was 0.730. Similar patterns were obtained when 120 resistant cells were assumed to pre-exist (r₀=0, δ₀=120).

### DETAILED DESCRIPTION

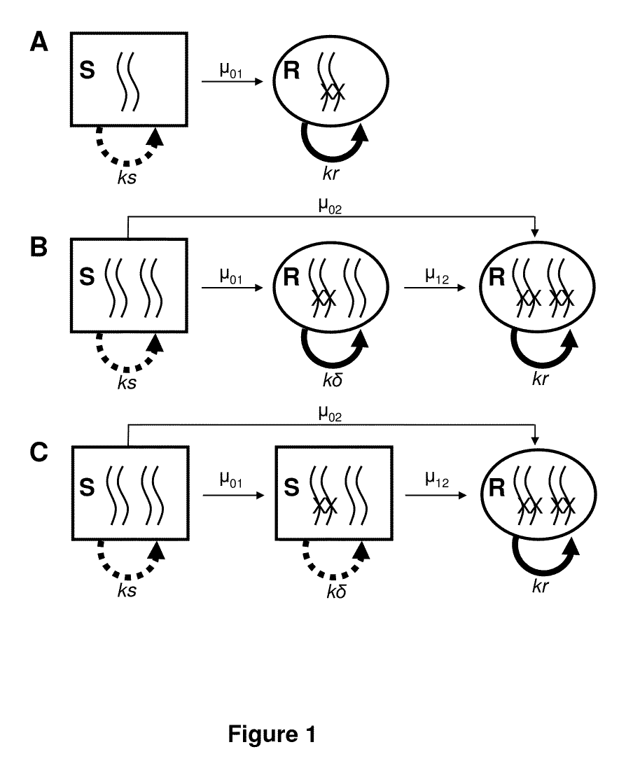

Methods for determining genetic mutation types, mechanisms (or “phenomena”) of acquired drug resistance, establishing genetic mechanisms or phenomena of acquired drug resistance, and methods for determining, monitoring, changing or predicting the response to a course of cancer therapy based on the genetic mechanism are provided herein. Tumor cells having acquired drug resistance are distinct from sensitive cells by their net cell growth rates at a given drug concentration. Further, acquired mutations may convert a sensitive cell into a resistant cell. These parameters may give rise to acquired drug resistance, which may arise through several genetic mutation types, such as those depicted in FIG. 1. As described in detail in the Examples below, experimental determination of the growth rate for sensitive cells and resistant cells using a particular drug concentration may be used alone or in combination with a computational or mathematical model to determine or identify the mutation type associated with an acquired resistance.

In one embodiment, a method for identifying or classifying a gene mutation associated with acquired drug resistance in a cancer is provided.

Cancer treatments that may be associated with a gene mutation that leads to acquired drug resistance may include, but are not limited to, tyrosine kinase inhibitor drugs and other protein kinase inhibitors including, but not limited to, AEE788, AMG 706, AMN107, ARRY-142886 (AZD6244), AZD2171, AZD0530, bevacizumab, BIBW 2992, BMS-354825, BMS-599626, CCI779, CEP-7055, cetuximab, CHIR-258, CI-1033, CP-724714, CP-547-632, dasatinib, E7080, erlotinib (Tarceva® or OSI774), fostamatinib, gefitinib (Iressa®), nilotinib, GW572016, GW786034, imatinib mesylate (ST157 or Gleevec®), lapatinib ditosylate (GSK572016), mubritinib, PD 0173074, PD 0325901, PKC412, PTK787, panitumumab, pazopanib, pegaptanib, rapamycin, ranibizumab, sorafenib, sunitinib (Sutent®), SU5416, SU11248, SU6668, trastuzumab, vandetanib, XL647, ZD6474, and analogs and derivatives thereof.

A “susceptible gene,” as described herein, is a gene in a cell that, once one or more gene mutations occur on that gene, the cell acquires drug resistance. The susceptible gene, with one or more copies in a cancer cell, may be already-mutated or wild type. The already-mutations include, but not limited to, deletion mutations, insertion mutations, inversion mutations, translocation mutations, duplication mutations, amplification mutations, or point mutations (e.g., substitution, small insertion, or small deletion). For example, the BCR-ABL fusion gene caused by translocation is the susceptible gene in CML and the already-mutation-activated EGFR gene caused by small deletion is the susceptible gene in NSCLC.

The one or more gene mutations may be a mutation or two or more different mutations. When the one or more gene mutations are a single mutation, said mutation may be found on one of the total copies, some of the total copies or on each of the total copies of the susceptible gene (N). When the one or more gene mutations are two or more different mutations, all of different mutations may be found on the same single copy of the total copy number of the susceptible gene, or the different mutations may be located on different copies of the total copy number of the susceptible gene. For example, two different mutations may be identified with a susceptible gene as being associated with acquired resistance. If a cell has a total of 2 copies of the susceptible gene, each copy may have a different mutation associated with it

Acquired resistance may occur as a result of any type of gene mutation on the susceptible gene, including, but not limited to, deletion mutations, insertion mutations, inversion mutations, translocation mutations, duplication mutations, amplification mutations, or point mutations (e.g., substitution, small insertion, or small deletion mutations). In some aspects, the mutation of a susceptible gene associated with the cancer treatment, such as those described above, is a point mutation. Additionally, the one or more a susceptible gene may include a single gene mutation. Point mutations in a gene targeted by a cancer drug are one of the most common mutation responsible for the development of acquired drug resistance. Acquired drug resistance may be associated with most general or targeted chemotherapeutic drugs or regimens used for treating cancer. In one embodiment, a kinase inhibitor may be associated with acquired drug resistance. Examples of genes targeted by a cancer drug that are susceptible to acquired resistance by the development of one or more point mutations include, but are not limited to, PDGFR-α, PDGFR-δ, EGFR, VEGFR, VEGFR1, VEGFR2, VEGFR3m HER-2 (also known as ErbB2), KIT, FLT3, c-MET, FGFR, FGFR1, FGFR3, c-FMS, RET, ABL, BCR-ABL, ALK, ARG, NTRK1, NTRK3, JAK2 and ROS.

In one embodiment, the cancer treatment used according to the methods described herein is imatinib. Imatinib inhibits several tyrosine kinases, including, but not limited to platelet-derived growth factor receptor (PDGFR) α and β, c-ABL, BCR-ABL, c-KIT, LCK, FGFR-1, VEGFR-1, 2, 3, and c-RAF. A point mutation, T315I, which substitutes a threonine residue with an isoleucine at amino acid position 315 of the BCR-ABL kinase domain, is a mutation that is associated with the development of acquired drug resistance with the use of imatinib. Other point mutations in the BCR-ABL kinase domain that may play a role in the development of acquired resistance as a result of imatinib treatment include, but are not limited to, Y253H, Y253F, F317L, M244V, G250E, Q252H, Q252R, E255K, M351T, E355G, F359V, V379I, L387M and H396R (Branford et al. 2002; Shah et al. 2002). In addition to point mutations associated with the BCR-ABL kinase domain, acquired drug resistance as a result of imatinib treatment may also be associated with a T6741 point mutation of the PDGFR-α gene in hypereosinophilic syndrome (HES) or a T670I point mutation of the KIT gene in gastrointestinal stromal tumors (GIST).

In other embodiments, the cancer treatment used according to the methods described herein is gefitinib and/or erlotinib are both inhibitors of EGFR tyrosine kinase activity. A point mutation, T790M, which substitutes a threonine residue with an methionine at amino acid position 790 of the EGFR kinase domain is one example of a mutation in the gene that is associated with the development of acquired drug resistance with the use of gefitinib and erlotinib. Other point mutations in the EGFR kinase domain that may play a role in the development of acquired resistance as a result of gefitinib or erlotinib treatment include, but are not limited to, L858R, H835L and R776C.

The mutations associated with acquired resistance according to the embodiments described herein may be present in cancer cells prior to any treatment, or may be secondary mutations that are acquired after a treatment has been administered.

The genes that are targeted by a cancer drug discussed above are associated with a variety of cancers. Therefore, gene mutations associated with acquired drug resistance in accordance with the embodiments of the methods described herein may apply to acquired drug resistance in any associated cancer including, but not limited to, CML (chronic myeloid leukemia), ALL (acute lymphoblastic leukemia), AML (acute myelogenous leukemia), T-ALL (T-Cell acute lymphoblastic leukemia), ALCL (acute lymphoblast cell leukemia), EMS (8p11 myeloproliferative syndrome), aCML (atypical chronic myelogenous leukemia), MM (multiple myeloma), T-lymphoma, MDS (myelodysplastic (syndrome), HES (hypereosinophilic syndrome), SM (systemic mastocytosis), and CMML (chronic myelomonocytic leukemia), IMT (inflammatory myofibroblastic tumor), NSCLC (non-small cell lung cancer), glioblastoma, SCCHN (squamous cell carcinoma of the head and neck), ovarian cancer, RCC (renal cell carcinoma), pancreatic cancer, colorectal cancer, breast cancer, lung cancer, GIST, seminoa, sarcomas, musculoskeletal tumors, gastric cancer, renal papillary carcinoma, malignant melanoma, PTC (papillary thyroid cancer), congenital fibrosarcoma, mesoblastic nephroma, secretory breast carcinoma, osteosarcoma, PAIS (pulmonary artery intimal sarcoma), DFSP (dermatofibrosarcoma protuberans), FMTC (familial medullary thyroid carcinoma), MEN-2B, radiation associated papillary thyroid cancer, astrocytoma, prostate cancer and renal cancer.

In some embodiments, the methods for described herein may include steps of determining the total number of copies of a gene that is susceptible to drug resistance (a “susceptible gene;” N) in a cancer cell and identifying a copy number of the susceptible gene that contain a point mutation associated with acquired drug resistance. Identification or determination of the copy number of a wild type or mutant gene may be accomplished by any suitable method, including, but not limited to, a gene sequencing method, a gene amplification method, single strand conformation polymorphism (SSCP), allele specific hybridization, primer extension, allele specific oligonucleotide ligation, gene chip hybridization assays, matrix assisted laser descoption ionization time of flight (MALDI-TOF) mass spectroscopy, fluorescent in situ hybridization (FISH) or a combination thereof.

Suitable gene sequencing techniques known in the art may include, but are not limited to, the Sanger method (e.g., chain terminator or dye terminator methods), high-throughput parallelized sequencing, and sequencing by hybridization, ligation, mass spectrometry, or electron microscopy.

Amplification of target gene sequences (DNA or RNA) in a cell or a tissue sample may be accomplished by any suitable method known in the art, such as transcription amplification, reverse transcription polymerase chain reaction (RT-PCR) amplification, quantitative PCR, restriction fragment length polymorphism PCR, ligase chain reaction, self-sustained sequence replication, arbitrarily primed polymerase chain reaction, selective amplification of target polynucleotide sequences, consensus sequence primed polymerase chain reaction, nucleic acid based sequence amplification, transcriptional amplification system, Q-Beta Replicase, rolling circle replication or any other nucleic acid amplification method, followed by the detection of the amplified molecules using suitable detection techniques known in the art.

In some embodiments, the methods described herein may include a step of determining a mutant copy number sufficient to cause acquired drug resistance (M). Such a determination may be accomplished experimentally or by using a computational model as described in the Examples below. The computational or mathematical models described herein may be used for in vitro determination of the mutant copy number sufficient to cause acquired drug resistance. Alternatively, the models may be used in a clinical setting to determine the mutant copy number sufficient to cause acquired drug resistance on an individual patient level.

In some embodiments, identification or classification of a gene mutation associated with acquired drug resistance is accomplished by comparing the total copy number of a susceptible gene (N) with the copy number of gene mutations of said susceptible gene sufficient to cause acquired drug resistance in a cell (M). A gene mutation may include, but is not limited to, a deletion mutation, an insertion mutation, an inversion mutation, a translocation mutation, a duplication mutation, an amplification mutation, a point mutation or a combination thereof. In one aspect, the mutation is a point mutation, such as those point mutations described herein. A type of gene mutation that is associated with acquired drug resistance may be a single copy mutation type, a dominant mutation type, an intermediate mutation type or a recessive mutation type.

In the case of a single copy mutation type, shown in FIG. 1A (mutation type A), only one copy of a susceptible gene is present in a tumor cell (N=1), which is equivalent to a haploid genome. When this single copy is mutated, the cell becomes resistant (M=1). For instance, in chronic phase CML, when the single copy of the BCR-ABL fusion gene is mutated to T315I, the individual cell becomes resistant to imatinib (Gorre et al 2001; Shah et al. 2002; Branford et al. 2002; Michor et al 2005).

However, in the advanced phase, a CML cell may contain two copies of BCR-ABL fusion gene (N=2), as in the case of blast crisis KCL-22 cells. This leads to two other potential types of gene mutations associated with acquired drug resistance (FIGS. 1B and C). With mutation type B, one mutant copy is sufficient for resistance (M=1), which is similar to a dominant inherited trait of a diploid genome. Thus, mutation type B is a dominant mutation type. On the other hand, with mutation type C, two mutant copies are required for resistance (M=2), which is similar to a recessive inherited trait of a diploid genome. Thus, mutation type C is a recessive mutation type.

The dominant and recessive mutation types (mutation types B and C, respectively) described herein may be identified or distinguished according to methods described herein. In one embodiment, a method for identifying a dominant mutation type in a cancer cell is provided. Such a method may include calculating a number of cells having 0, 1 or 2 mutant copies of a gene susceptible to acquired resistance at a series of predetermined time intervals. In some embodiments, said calculations may be accomplished by using a computational or mathematical model as described in the Examples below. Further, in some embodiments, a computational or mathematical modeling system may be used in accordance with the methods described herein. The modeling system may include a computer system that calculates one or more equations (e.g., Equations 1, 2, 3a, 3b, 3c, described below) associated with the computation or mathematical model. The results of said calculation may be used to identify a gene mutation type associated with acquired drug resistance.

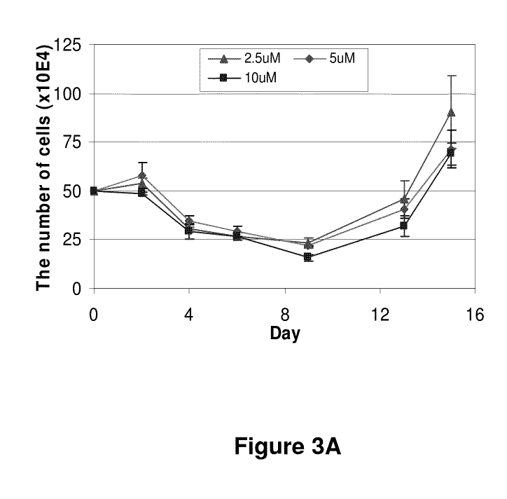

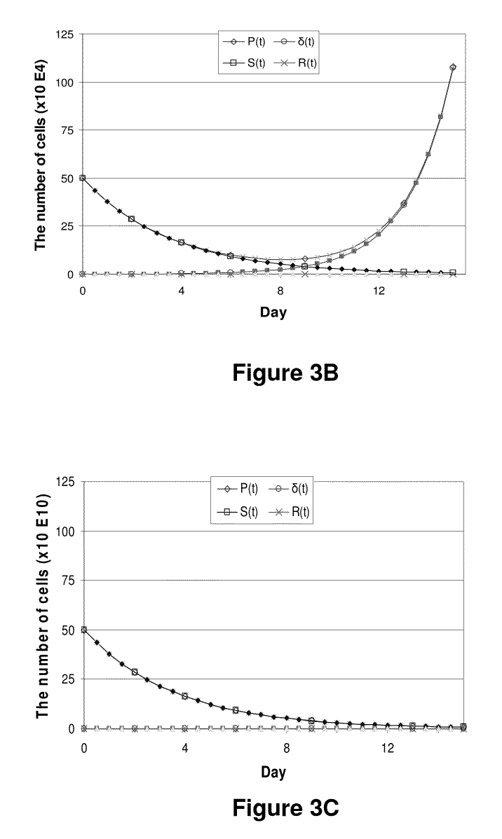

The computational or mathematical model may include Equations 3a, 3b and 3c below, which correspond to a calculation of cells having 0, 1 or 2 mutant copies of the mutant gene, respectively. These equations (3a, 3b and 3c) may then be used to generate a series of simulated growth kinetics graphs for cells having 0, 1 or 2 mutant copies at each time interval. For example, as shown in FIG. 3B, the Equations 3a, 3b and 3b are calculated at an interval of 6 hours for a total of 15 days and superimposed on a single graph. Based on the data points calculated at each time interval using the equations, the graph may generated by any suitable statistical method known in the art, for example, a nonlinear regression analysis. In one aspect, the graphs or series of graphs may be a best-fit nonlinear regression model based on the equations described herein.

A constant growth rate and a constant mutation rate were assumed when calculating Equations 3a, 3b and 3c. In some embodiments, the constant growth rate and mutation rates are determined experimentally by any suitable method, including, but not limited to those methods described herein. In some aspects, the constant growth rate may be determined by counting cells treated with a drug associated with acquired resistance for a given unit of time, as illustrated by Equation 2 below. In other aspects, the constant mutation rate may be determined by a soft agar colony formation assay, the results of which are used to calculate a mutation rate according to Equation 1 below.

In some embodiments, the series of simulated growth kinetics graphs is compared to a set of experimentally determined growth kinetics data to determine whether a cancer cell acquires resistance to a drug through a dominant mutation type. In one aspect, the cancer cell is determined to acquire resistance through a dominant mutation type when the series of simulated growth kinetics graphs fit the experimentally determined growth kinetics data. The series of simulated growth kinetics graphs and the experimentally determined growth kinetics data may be determined to “fit” by a qualitative or quantitative comparison. In some aspects, a quantitative comparison may be made by calculating the “goodness of fit” between the simulated growth kinetics graphs generated using the computation or mathematical model described herein and the experimentally determined growth kinetics data. Goodness of fit may be calculated by any suitable method, such as the coefficient of determination (R²), which is a statistical measure of how well the regression line generated by the simulated model fits or approximates the actual experimental data. The R² is the fraction of the variation that is shared between X and Y, which ranges between 0 and 1.0, with 0 representing an absence of fit and 1.0 being an exact fit.

In some embodiments, the experimentally determined growth kinetics data may be determined by any suitable method, for example, a cell viability assay after treatment with a drug associated with acquired resistance as described below. FIG. 3A illustrates the results of such a viability assay. In one embodiment, the acquired imatinib resistance mutation types B and C were validated in blast crisis KCL-22 cells by cell culture experiments, direct sequencing analysis, and mathematical simulations using computational models described in the Examples below. To identify a dominant genetic resistance mutation type, described as mutation type B above, imatinib resistance of blast crisis in chronic myeloid leukemia cell line KCL-22 was analyzed as described below. Each KCL-22 cell contains two copies of Philadelphia chromosome t(9; 22) harboring the BCR-ABL fusion gene. In this mutation type, it is sufficient for an individual KCL-22 cell to become imatinib resistant when one of these two copies is mutated to T315I.

When cancer cells contain a multi-ploid genome, there may be other types of mutations responsible for drug resistance. For instance, a susceptible gene, such as the deletion-activated EGFR gene, contains 3 copies per cell, presumably due to gene amplification or cell fusion (Pawelek 2005; Friedl 2005; Lu & Kang 2009). Drug resistance may occur when one of the 3 mutation-activated copies mutates (dominant resistance) (Mutation type D in Table 1). However, 2 or more of the 3 copies may need to be mutated before the cell exhibits recessive resistance or an intermediate genetic drug resistance, which is a mutation type that falls between dominant and recessive mutation types (Mutation types E and F in Table 1 below).

TABLE 1
Genetic mechanisms of drug resistance

| | Susceptible genes | | | | |
|---|---|---|---|---|---|
| | Number of | | Minimum | | |
| Mutation | the genes | Gene copies | mutated copies | | |
| Type | involved | per cell (N) | needed (M) | Comment | Example |
| A | 1 | 1 | 1 | | Chronic phase |
| | | | | | CML |
| B | 1 | 2 | 1 | Dominant | Advanced phase |
| | | | | | CML |
| C | 1 | 2 | 2 | Recessive | |
| D | 1 | ≧3 | 1 | Dominant | NSCLC treated |
| | | | | | with gefitinib or |
| | | | | | erlotinib |
| E | 1 | ≧3 | 2 | M < N, | |
| | | | | Intermediate | |
| F | 1 | ≧3 | ≧3 | M = N, | |
| | | | | Recessive | |

In chronic phase CML patients, the individual cell becomes resistant to imatinib when the single copy of the BCR-ABL fusion gene is mutated to T315I (Gorre et al 2001; Shah et al. 2002; Branford et al. 2002; Michor et al 2005). However, in advanced phases CML, cells may contain two copies of the BCR-ABL fusion gene, as in the case of blast crisis KCL-22 cells. When KCL-22 cells were treated with 2.5, 5 or 10 μM imatinib, equivalent to the effective concentrations in clinical treatments (Peng et al. 2005), cells underwent initial apoptosis but relapsed after eight to nine days, due to the emergence of the resistant KCL-22M cells.

KCL-22M cells bear a single type of point mutation, T315I, in the BCR-ABL kinase domain as determined by PCR and Sanger sequencing to analyze cDNA and genomic DNA. It was then determined whether KCL-22M cells contain either one or two copies of the T315I mutation per cell, which was not previously known (Yuan et al. 2010). This information was then used in the model described herein for determining the genetic mutation type associated with the T315I mutation.

In some embodiments, methods for selecting or modifying a treatment or treatment regimen for a subject having cancer based on predicting the response to or monitoring the response to said cancer treatment or treatment regimen are provided. A “response to a cancer treatment or treatment regimen” refers to the clinical benefit imparted to a subject suffering from a disease or condition (e.g., cancer) from or as a result of the cancer treatment or treatment regimen. A clinical benefit includes a complete remission, a partial remission, a stable disease without progression, progression-free survival, disease free survival, improvement in the time-to-progression of the disease, improvement in the time to death, or improvement in the overall survival time of the patient from or as a result of the treatment or treatment regimen. There are criteria for determining a response to therapy and those criteria allow comparisons of the efficacy to alternative treatments (see Slapak and Kufe, Principles of Cancer Therapy, in Harrison's Principles of Internal Medicine, 13^{th} ed., eds. Isselbacher et al., McGraw-Hill, Inc., 1994).

In some aspects, the methods for selecting or modifying a treatment or treatment regimen may include identifying a cancer as having one or more gene mutations associated with acquired resistance to one or more cancer drugs. The one or more gene mutations may be a deletion mutation, an insertion mutation, an inversion mutation, a translocation mutation, a duplication mutation, an amplification mutation, a point mutation or a combination thereof. In one aspect, the mutation is a point mutation, such as those point mutations described herein.

In other aspects, the methods for selecting or modifying a treatment or treatment regimen may include determining or identifying a genetic drug resistance mutation type, such as a single gene, a dominant, an intermediate or a recessive genetic drug mutation type for each of the one or more point mutations. When a dominant mutation type is identified in a cancer cell, drug resistance may arise earlier and more frequently. Thus, the copy number of a susceptible diploid or multi-ploid gene due to cell fusion, gain of chromosomes, or gene amplification (Pawelek 2005; Margolis 2005) may play an important role in drug resistance and serve as a marker to guide therapeutic decisions. For example, a subject or patient for whom a certain generally used therapy is ineffective may be identified at an early stage and the subject may be treated with an alternative therapy that is adapted to the subject's acquired resistance susceptibility and response to therapies without having to go through a painful and possible detrimental therapy. In other words, the subjects who do not benefit from a treatment or whom a treatment would be detrimental are identified.

In some embodiments, when a genetic drug resistance mutation type is identified for a particular susceptible gene in response to a particular cancer treatment, the mutation type may be used in combination with other genetic drug resistance mutation types that have been or will be identified for one or more other susceptible genes in response to one or more cancer treatments. In this case, all identified genetic mutation types may serve to establish a set of standards from which a drug resistance phenotype may be determined. A drug resistance phenotype, as used herein, refers to a measure of susceptibility to one or more drugs in a subject having cancer based on the presence of one or more identified mutant susceptible genes in a biological sample that contains or may contain cancer cells or their nucleic acid components (e.g., blood, serum, plasma, lymph, cerebrospinal fluid, bone marrow, tumor tissue) from the subject. This phenotype provides a determination of which cancer treatments will be effective in eliciting a response in a patient and which will not.

Further, in other aspects, the methods for selecting or modifying a treatment or treatment regimen may include selecting or modifying a treatment or treatment regimen for a subject having cancer based on the determination or identification of a genetic drug resistance mutation type.

In some aspects, when a susceptible gene is associated with or identified as having a dominant mutation type, it is predicted that the response to the treatment or treatment regimen associated with the dominant mutation type will be ineffective or ineffective after a short period of treatment. Thus, said treatment or treatment regimen should be avoided or stopped to limit any resistance effects of the drug and an alternative treatment or treatment regimen should be started. In the case of a cancer that is associated with a dominant mutation type of resistance with the treatment of imatinib, such as CML, the identification of a dominant mutation type would allow clinicians to consider limiting treatment of CML to using imatinib for short periods of time or to alternative treatments and treatment regimens that are not associated with the T315I mutation.

In other aspects, when a susceptible gene is associated with or identified as having a recessive mutation type, it is predicted that the response to the treatment or treatment regimen associated with the recessive mutation type will be effective for a longer time than for a dominant mutation type. Thus, administering the treatment or treatment regimen associated with the recessive mutation type may be selected initially, then a response to the treatment or treatment regimen may be monitored for any changes (i.e., increases) in the mutant copy number as a result of the treatment or treatment regimen. Once an increase in the mutant copy number is detected, an alternative treatment or treatment regimen should be selected.

Alternative treatments that may be used in accordance with the embodiments described herein may include, but are not limited to, drugs, chemotherapeutic agents (e.g., alkylating agents, antimetabolites, anti-tumor antibiotics, plant alkyloids, topoisomerase inhibitors, mitotic inhibitors hormone therapy, targeted therapeutics and immunotherapeutics), therapeutic antibodies and antibody fragments (e.g., alemtuzumab, bevacizumab, cetuximab, edrecolomab, gemtuzumab, ibritumomab tiuxetan, panitumumab, rituximab, tositumomab, and Trastuzumab), toxins (ricin, abrin, ribonuclease (RNase), DNase I, Staphylococcal enterotoxin-A, pokeweed antiviral protein, gelonin, diphtheria toxin, Pseudomonas exotoxin, and Pseudomonas endotoxin), radioisotopes (e.g, ³²P, ⁸⁹Sr, ⁹⁰Y. ^{99m}TC, ⁹⁹Mo, ¹³¹I, ¹⁵³Sm, ¹⁷⁷Lu, ¹⁸⁶Re, ²¹³Bi, ²²³Ra and ²²⁵Ac), enzymes (e.g., enzymes to cleave prodrugs to a cytotoxic agent at the site of the tumor), nucleases, hormones, immunomodulators, antisense oligonucleotides, chelators, boron compounds, photoactive agents and dyes.

The above-mentioned treatments may be used alone or in combination with each other or in combination with other treatment modalities in an alternative treatment regimen. An alternative treatment regimen according to the embodiments described herein includes, but is not limited to, combined modality chemotherapy (i.e., the use of drugs with other cancer treatments, such as radiation therapy or surgery) or combination chemotherapy (i.e., the use of different chemotherapeutic agents combined simultaneously to enhance their effectiveness).

The following examples are intended to illustrate various embodiments of the invention. As such, the specific embodiments discussed are not to be construed as limitations on the scope of the invention. It will be apparent to one skilled in the art that various equivalents, changes, and modifications may be made without departing from the scope of invention, and it is understood that such equivalent embodiments are to be included herein. Additionally, although the methods described herein generally refer to genetic mutation types responsible for acquired drug resistance, the cause of acquired drug resistance may also be referred to as a genetic “mechanism” or “phenomena.” Further, all references cited in the disclosure are hereby incorporated by reference in their entirety, as if fully set forth herein.

### EXAMPLES

### Example 1

### Analysis of Blast Crisis CML Cell Line

As described in the example below, a dominant genetic mutation type of acquired drug resistance has been identified and validated. In this mutation type, it is sufficient for an individual cancer cell to become drug resistant when one copy of a susceptible diploid or multi-ploid genome is mutated. Thus, the copy number of a susceptible diploid or multi-ploid gene due to cell fusion, gain of chromosomes, or gene amplification may play an important role in drug resistance and serve as a marker to guide therapeutic decisions as described above.

Materials and Methods

Cytogenetic Characteristics of KCL-22 Cells.

KCL-22, a blast crisis CML cell line, was purchased from the German Collection of Cell Cultures, Braunschweig, Germany. The karyotype of this cell line was analyzed and was determined to be 51, X, del (X)(p11.2p22.3), +der(1; 10)(q10; p10), +6, +8, +8, t(9; 22)(q34.1; q11.2), der(17; 19)(q10; q1), +19, i(21)(q10), +der(22)t(9; 22). Two copies of Philadelphia chromosomes t(9; 22) were identified in each KCL-22 cell. The resistant cells, designated as KCL-22M, showed the same cytogenetic characteristics (Yuan et al. 2010).

Resistance Assay.

Half a million KCL-22 cells were seeded in 1 ml of RPMI 1640 medium with 10% fetal bovine serum (Hyclone, SH30071.03) per well in triplicate and treated with 2.5, 5 or 10 μM imatinib (STI-571). For KCL-22M cells, 100,000 cells were cultured in 1 ml of medium per well in triplicate. Aliquots of cells were removed at given time points, the number of cells was counted on a hematocytometer, and cell viability was assessed by trypan blue exclusion.

Soft Agar Colony Formation Assay.

For colony formation, a standard two-layer soft agar culture was used at 2.5, 5 or 10 μM imatinib. One million KCL-22 cells were seeded per well in triplicate and incubated for 3 weeks. Plates were then stained with 0.005% Crystal Violet for 1 hour, and colonies were scored by microscope.

Sanger Sequencing Analysis of Genomic DNA from Individual Resistant Clonal Cells.

To determine whether KCL-22M cells contain either one or two copies of the T315I mutation per cell, an intron 5 primer 5′-GAGCCACGTGTTGAAGTCCT-3′ (SEQ ID NO:1) and an exon 6 primer 5′-TTTGTAAAAGGCTGCCCGGC-3′ (SEQ ID NO:2) were designed to span ABL exon 6 within the ABL kinase domain. In this design, two copies of the BCR-ABL fusion gene and one copy from the ABL gene of each cell were amplified by PCR. After purification by Qiagen MinElute PCR purification kit, the PCR product was sequenced with each primer using ABI 3730 fluorescent DNA sequencer and BigDye terminator chemistry V3.1 (Applied Biosystems). Sequencher software (Gene Codes) was used to identify the mutation on chromatograph.

Acquired Imatanib Resistance in KCL-22M Cells is Acquired Through a Dominant Mutation Type of Resistance

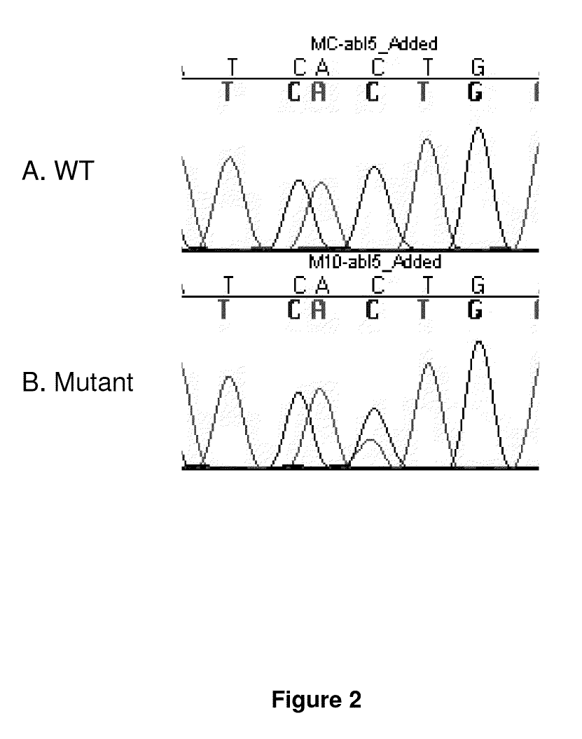

To determine if KCL-22M cells contain one or two copies of the T315I mutation per cell, 20 genomic DNA pooled templates were sequenced from individual KCL-22M cell clones. It was found that each clone carried one copy of the T315I mutation per cell (FIG. 2). KCL-22M cell clones containing two T315I copies per cell were not observed, likely because two copies of the T315I mutation occur at a low or negligible rate.

Furthermore, no other mutations were identified in the kinase domain or the other functionally important oligomerization and SH3/2 domains of the BCR-ABL fusion gene (Yuan et al. 2010). These results indicate that the acquired imatinib resistance in KCL-22M cells is predominantly through one copy of the T315I mutation, i.e., a dominant mutation type of resistance.

In addition, it has been shown that KCL-22M cells have many similar characteristics as KCL-22 cells (Table 2) (Yuan et al. 2010).

TABLE 2
Comparison of other features between KCL-22 and KCL-22M cells

| Feature | KCL-22 | KCL-22M | Methods |
|---|---|---|---|
| BCR-ABL mRNA | Ct = 15, the same level | Ct = 15, the same level | Real-time PCR |
| BCR-ABL protein | Decrease with higher | Constant level with | Western blot |
| | concentration of imatinib | or without imatinib | |
| Cytogenetics | Two Philadelphia | Two Philadelphia | 24-color special karyotyping |
| | chromosomes ^{a} | chromosomes ^{a} | and three-color FISH |
| Cell cycle | G1 56.8%, S 37.8%, | G1, 45.0%, S 42.1%, | Flow cytometer |
| | G2/M 5.4% | G2/M 12.9% | |

### Example 2

### Computational and Mathematical Models for Determination of the Genetic Mutation Type of Imatinib Resistance of a Blast Crisis CML Cell Line

To quantitatively discriminate between mutation types B and C (FIGS. 1B and C) for imatinib resistance, mathematical models were developed for simulation using at least the following assumptions: 1) that under a given drug concentration, KCL-22 cells and KCL-22M cells grow at constant but different rates during the exponential growth phase; and 2) that the T315I mutation rate is constant.

The constant growth and mutation rates used in the mathematical models described below were measured using resistance assays and soft agar colony formation assays, respectively (Table 3).

TABLE 3
Experimental growth rates and mutation rates

| | Growth rate | Growth rate | |
|---|---|---|---|
| Imatinib | of KCL-22 | of KCL-22M | T315I mutation rate on |
| (μM) | cells, kₛ ^{a} | cells, kᵣ ^{b} | soft agar, μ ^{c} |
| 2.5 | −0.245 | 0.730 | 2.40 × 10⁻⁴ ± 2.89 × 10⁻⁵ |
| 5 or 10 | −0.225 | 0.630 | 1.25 × 10⁻⁴ ± 1.94 × 10⁻⁵ |

^{a} kₛ was estimated from the number of viable KCL-22 cells counted between days 2 and 4 assuming that the cells grow exponentially (negatively) in resistance assay with imatinib.
^{b} kᵣ was estimated from the number of viable KCL-22M cells counted between days 2 and 4 assuming that the cells grow exponentially (positively) with imatinib.
^{c} μ was estimated by clone formation assay with one unit assigned to be the number of the T315I mutations incurred per BCR-ABL fusion gene with their 95%

In addition, to show transfer rates in the network, a probability matrix of μ₀₁, μ₀₂, μ₁₂, as well as μ₀₀ and μ₁₁ were introduced from conversion of the observed T315I mutation rates on soft agar colony formation assay. For example, μ₀₁ is the transfer rate from the sensitive cells with zero mutant copies to the cell cells with one mutant copy) (Table 4). The exponential growth functions (Eq. 1, below) were chosen because they fit the negative cell growth of the blast crisis CML cells the best.

TABLE 4
Matrix of transfer rates among the three types of cells in FIG. 1B and C^{a}

| Imatinib | Transfer rate | | | | |
|---|---|---|---|---|---|
| (μM) | μ₀₀ | μ₁₁ | μ₀₁ | μ₀₂ | μ₁₂ |
| 2.5 | 0.9995200576000 | 0.9997600000000 | 4.80 × 10⁻⁴ | 5.76 × 10⁻⁸ | 2.40 × 10⁻⁴ |
| 5 or 10 | 0.9997500156250 | 0.9998750000000 | 2.50 × 10⁻⁴ | 1.56 × 10⁻⁸ | 1.25 × 10⁻⁴ |

^{a}Calculated from the T315I mutation rates on soft agar colony formation assay and Eq. 2 below, which are 2.40 × 10⁻⁴ at 2.5 μM imatinib, and 1.25 × 10⁻⁴ at 5 or 10 μM imatinib, respectively. One unit is assigned to be the number of the mutations incurred per BCR-ABL fusion gene.

Mathematical Models for Quantification of Genetic Models.

For simplicity, the simulations described herein focus on the exponential phase of cell growth. Three assumptions were taken into account: 1) at a given drug concentration, sensitive cells grow at their constant net growth rate, kₛ, and resistant cells at their constant rate, kᵣ, where one day is used as the unit of time (Eq. 1, below); 2) mutations which may convert a sensitive cell into a resistant cell occur at a constant rate μ, (Eq. 2, below); and 3) resistant cells may or may not exist before treatment.

$$\begin{matrix}μ = \frac{ \begin{matrix}the number of mutations \\ that occurred after 1 unit of time\end{matrix} }{\begin{matrix}the total number of gene copies \\ in the cell population\end{matrix}} & ( Eq . 1 ) \\ k = \frac{the number of new cells after 1 unit of time}{the number of cells before that unit of time} & ( Eq . 2 )\end{matrix}$$

Next, let S(t), δ(t) and R(t) represent the number of cells that have 0, 1, or 2 mutant copies, let μ_{ab} represent the mutation rate from a to b copies of the mutant gene, and let each population have associated with it initial values S₀, δ₀, and R₀ and net growth rates kₛ, k_{δ}, and kᵣ. The three equations below, Eq. 3a, 3b and 3c represent three populations of S(t), δ(t) and R(t), respectively.

$$\begin{matrix} S ( t ) = {{s}_{0} ( 1 + {k}_{s} )}^{t} {μ}_{00}^{t} & ( Eq . 3 a ) \\ δ ( t ) = {{δ}_{0} ( 1 + k δ )}^{t} {μ}_{11}^{t} + {μ}_{01} {\int }_{0}^{t} S ( x ) {( 1 + k δ )}^{t - x} {μ}_{11}^{t - x} d x & ( Eq . 3 b ) \\ R ( t ) = {{r}_{0} ( 1 + {k}_{r} )}^{t} + {μ}_{02} {\int }_{0}^{t} S ( x ) {( 1 + {k}_{r} )}^{t - x} d x + {μ}_{12} {\int }_{0}^{t} δ ( x ) {( 1 + {k}_{r} )}^{t - x} d x & ( Eq . 3 c )\end{matrix}$$

Next, let μ_{ab} represent the mutation rate from a to b copies of the mutant gene of a total of n copies, which can be reduced by using a Binomial distribution (Eq. 4). It is assumed that mutations are irreversible (a≦b) and occur independently and constantly.

μ_{ab}=P(a→b)=(_{b-a}^{n-a})m^{b-a}(1−m)^{n-b} (Eq. 4)

In the case of two copies of the BCR-ABL fusion gene, μ₀₁, μ₀₂, μ₁₂, as well as μ₀₀ and μ₁₁ are obtained based on the T315I mutation rates on soft agar colony formation assay; the units of the mutations are defined here as per BCR-ABL fusion gene (To show mutation rates in the networks, μ₀₀ and μ₁₁ were introduced. For instance, μ₀₀ is the transfer rate from the sensitive cell with 0 copies of the mutant to the same sensitive cells with 0 copies of the mutant).

Statistical Analysis.

Mathematical models corresponding to genetic models were done according to the Akaike Information Criterion (Akaike 1974), which penalizes models for additional parameters that are not sufficiently effective in improving the fit, were used to select the best model, and thus supports the underlying hypothesis that it represents. Model differences in goodness of fit largely occurred when disease recurrence became obvious at an early recurrence day 13.

Simulated Cell Growth Kinetics of Blast Crisis KCL-22 Cell Line

Using the experimental growth and mutation rates described above for 2.5, 5 or 10 μM imatinib, cell growth kinetics were simulated using equations 3a, 3b and 3c to reveal the underlying mutation type that is most consistent with the data obtained. Simulations were performed with and without pre-existing resistant cells.

At 2.5 μM imatinib, it was determined whether the simulated cell growth patterns from mutation type B fit the observed kinetics (FIG. 3A). On day 13, at an early stage of relapse, the number of viable cells between the experiment and simulation was compared at each time point using a t-test (p=0.27 and 0.37 without and with pre-existing mutations, respectively), supporting mutation type B (FIG. 1B). In contrast, the simulated cell growth patterns from mutation type C did not fit the data on day 13 (p=0.016 both with and without pre-existing mutations).

At higher concentrations of 5 or 10 μM imatinib, the simulated cell growth patterns from mutation type B fit the observed kinetics much better than those from mutation type C (on day 13 p>0.02 and 0.03 with and without pre-existing mutations with mutation type B vs. p<0.01 and 0.01 with mutation type C). This result is compatible with that at a dose of 2.5 μM.

### Simulated Mutation Rates of Blast Crisis KCL-22 Cell Line

Next, the mutation rates from the experimental growth rates were simulated (Table 5). According to mutation type B, the simulated values are much closer to the observed mutation rates obtained from the colony formation assay. Again, the data supports mutation type B as opposed to C (FIGS. 3B and C).

TABLE 5
Simulated mutation rates from the experimental growth rates

| | Observed | | | | |
|---|---|---|---|---|---|
| | | Growth | Growth | Simulated mutation rate^{a} | |
| Assumed | | rate of | rate of | No pre- | Pre- |
| Mutation | Imatinib | KCL-22 | KCL-22M | existing | existing |
| Type | (μM) | cells, kₛ | cells, kᵣ | mutations | mutations^{b} |
| B | 2.5 | −0.245 | 0.730 | 2.02 × 10⁻⁴ | 1.42 × 10⁻⁴ |
| C | | | | 1.48 × 10⁻² | 1.19 × 10⁻² |
| B | 5 or 10 | −0.225 | 0.630 | 3.38 × 10⁻⁴ | 2.46 × 10⁻⁴ |
| C | | | | 1.97 × 10⁻² | 1.57 × 10⁻² |

^{a}one unit is the number of the T315I mutations incurred per BCR-ABL fusion gene per day.
^{b}the number of pre-existing mutations are assumed to be 120 and 62.5 from half a million of KCL-22 cells at 2.5 μM and 5 or 10 μM imatinib, respectively.

### Example 3

### Analysis of NSCLC

Materials and Methods

Collection of Cancer Samples.

Tissues from 28 NSCLC patients who were diagnosed at early stages (IA, IB, IIA, and IIB) were collected. Cancer tissues and their paired marginal normal tissues were sectioned in surgery and immediately frozen at −70° C. Cancer tissues contained sufficient portion of tumor cells, typically ≧40%, and normal tissues had no tumor cells initially judged by a pathologist.

Fluorescence In Situ Hybridization (FISH) Analysis.

After frozen tissues were formalin fixed and paraffin embedded, 5 μm thick sections were cut and stained with hematoxylin and eosin. Morphological analyses were performed to determine the ratio of tumor area to the total area on slides by two investigators.

Cover slips were removed in xylene and slides were fixed in Carnoy's fixative (3:1 methanol:acetic acid) for 30 min. Slides were then placed in 2×SSC for 10 min, followed by 0.05% pepsin in 10 mM HCl at 37° C. for 10 min.

After dehydration through ethanol series, the Vysis EGFR/CHP 7 probe (cat#30-191053) (Abbott Molecular, Abbott Park, Illinois) was applied. The probe and section were co-denatured at 80° C. for 5 minutes. After overnight incubation at 37° C. post wash was performed per manufacturer's direction.

Images were acquired using Bioview D3 image analyzer (Bioview) to capture the cell morphology. For each probe set, sixty cells were examined for each case by two independent scorers. Their average copies per tumor cell were normalized by positive normal standards, and then rounded to the nearest integer.

PCR and Sanger Sequencing for in-Frame Mutations in Exon 19 and for Missense Mutations in exon 21 of the EGFR Gene.

Primers were designed to amplify and sequence exon 19 (Forward: 5′CACAGCCCCAGTGTCCCTCACC3′ (SEQ ID NO:3); Reverse: 5′GGATGTGGAGATGAGCAGGGTCTA3′; (SEQ ID NO:4)) and exon (Forward: 5′TGGCATGAACATGACCCTGAAT3′ (SEQ ID NO:5); Reverse: 5′GCATCCTCCCCTGCATGTGTTA3′ (SEQ ID NO:6) of the EGFR gene. Each PCR mixture contained a total volume of 25 μl: 50 mM KCl, 10 mM Tris/HCl (pH 8.3), 1.5 mM MgCl₂, 200 μM each dNTPs, 0.1 μM each primer, 1 U of TaqGold DNA polymerase (Applied Biosystems), and 20 ng of genomic DNA. The cycling entailed denaturation at 94° C. for 15 seconds, annealing at 55° C. for 30 second, and elongation at 72° C. for 1 minute for 35 cycles. Before the cycling, 94° C. for 10 minutes was applied to activate TaqGold DNA polymerase (Roche).

The PCR product was purified using Amocon50 to remove the unincorporated primers and dNTPs. Two nanogram of the PCR product was sequenced using ABI 3730 fluorescent DNA sequencer and BigDye terminator chemistry V1.1 (Applied Biosystems) with the above PCR primers. Sequencher software (Gene Codes) was used to identify a point somatic mutation when its mutant peak had ≧18% of the wild-type peak height, equivalent to when 30% of diploid cells contains a copy of the mutation.

Genetic Mutation Types D to F with Multi-Ploid Genome

In non-small cell lung cancer (NSCLC) patients, erlotinib or gefitinib initially elicit dramatic responses through inhibition of somatic mutation-activated EGFR in cancer cells (Sharma et al. 2007; Ciardiello & Tortora 2008). However, many patients develop acquired resistance due to a second point mutation, such as T790M, in the mutation-activated EGFR gene (Bell et al. 2005).

Using PCR and Sanger sequencing from 28 early stage NSCLC patients, five such somatic activated mutations were identified in exon 19 of the EGFR gene that target the tyrosine kinase domain (Table 6). In addition, the mutations were only found in the cancer tissues but not in the paired normal tissues, indicating that the mutations are cancer-specific. Furthermore, using FISH, the total copy number of the EGFR gene, mutation-activated and wild-type, were also measured. Thus, the copy number of the mutation-activated EGFR gene was estimated per cell.

TABLE 6
The susceptible mutation-activated EGFR gene and their copy number
per cell

| | | | | | | | % of | |
|---|---|---|---|---|---|---|---|---|
| | | | | | | | tumor | Estimated |
| | FISH | | PCR and Sequencing | | | | cells | mutation- |
| | Total | Total CH7 | | | | Mut to | within | activated |
| | EGFR | centromer | | | AA | WT | tissue | gene |
| Patient | copies/cell | copies/cell | Type | Position^{a} | change | peak^{b} | specimen | copies/cell^{c} |
| A3 | 8 | 6 | 18-nt del | 155,746-63 | In frame | 50% | 65% | 3 |
| A9 | 6 | 6 | 15-nt del | 155,742-56 | In frame | 100% | 80% | 3 |
| C9 | 6 | 4 | 15-nt del | 155,741-55 | In frame | 40% | 55% | 2 |
| E3 | 2 | 2 | 15-nt del | 155,742-56 | In frame | 50% | 70% | 1 |
| E5 | 2 | 2 | 15-nt del | 155,741-55 | In frame | 30% | 75% | 1 |
| A7 | 5 | 4 | T to G | 172,791 | Leu to | 45% | 70% | 2 |
| | | | | | Arg | | | |
| C5 | 2 | 2 | T to G | 172,791 | Leu to | 30% | 70% | 1 |
| | | | | | Arg | | | |

^{a}Numbered according to NC_000007. Region: 55054219 . . . 55242525
^{b}The ratio of mutant to WT peaks was estimated from both directions on sequencing data.
^{c}estimated from the total EGFR copy number per cell, the relative peak height of the mutations, and % of
tumor cells within tissue specimen.

With multi-ploid genome of the mutation-activated EGFR gene, there are other genetic mutation types for the acquired resistance to erlotinib or gefitinib (Mechanisms D to F in Table 1). Using FISH, PCR and Sanger sequencing, each tumor cell was found to contain three copies of the mutation-activated EGFR gene in NSCLC patients A3 and A9 (Table 6). Drug resistance may occur when one of the three mutation-activated copies contains a second T790M mutation, showing a super-dominant nature (Mechanism D in Table 1). However, two or more of the three mutation-activated copies may be needed to mutate before the cell exhibits resistance with 3/3 recessive patterns or 2/3 intermediate pattern (Mechanisms E and F in Table 1).

### REFERENCES

The references, patents and published patent applications listed below, and all references cited in the specification above are hereby incorporated by reference in their entirety, as if fully set forth herein.
1. Luria S E, Delbruck M (1943) Mutations of Bacteria from Virus Sensitivity to Virus Resistance. Genetics 28:491-511
2. Eaves C, Cashman J, Eaves A (1998) Defective regulation of leukemic hematopoiesis in chronic myeloid leukemia. Leukemia research 22:1085-1096
3. Radivoyevitch T, Kozubek S, Sachs R K (2002) The risk of chronic myeloid leukemia: can the dose-response curve be U-shaped? Radiation research 157:106-109
4. Radivoyevitch T, Kozubek S, Sachs R K (2001) Biologically based risk estimation for radiation-induced CML. Inferences from BCR and ABL geometric distributions. Radiation and environmental biophysics 40:1-9
5. Niederwieser D, Maris M, Shizuru J A, Petersdorf E, Hegenbart U, Sandmaier B M, Maloney D G, Storer B, Lange T, Chauncey T, Deininger M, Ponisch W, Anasetti C, Woolfrey A, Little M T, Blume K G, McSweeney P A, Storb R F (2003) Low-dose total body irradiation (TBI) and fludarabine followed by hematopoietic cell transplantation (HCT) from HLA-matched or mismatched unrelated donors and postgrafting immunosuppression with cyclosporine and mycophenolate mofetil (MMF) can induce durable complete chimerism and sustained remissions in patients with hematological diseases. Blood 101:1620-1629
6. Gorre M E, Mohammed M, Ellwood K, Hsu N, Paquette R, Rao P N, Sawyers C L (2001) Clinical resistance to STI-571 cancer therapy caused by BCR-ABL gene mutation or amplification. Science 293:876-880
7. Shah N P, Nicoll J M, Nagar B, Gorre M E, Paquette R L, Kuriyan J, Sawyers C L (2002) Multiple BCR-ABL kinase domain mutations confer polyclonal resistance to the tyrosine kinase inhibitor imatinib (STI571) in chronic phase and blast crisis chronic myeloid leukemia. Cancer cell 2:117-125
8. Branford S, Rudzki Z, Walsh S, Grigg A, Arthur C, Taylor K, Herrmann R, Lynch K P, Hughes T P (2002) High frequency of point mutations clustered within the adenosine triphosphate-binding region of BCR/ABL in patients with chronic myeloid leukemia or Ph-positive acute lymphoblastic leukemia who develop imatinib (STI571) resistance. Blood 99:3472-3475
9. Pao W, Miller V, Zakowski M, Doherty J, Politi K, Sarkaria I, Singh B, Heelan R, Rusch V, Fulton L, Mardis E, Kupfer D, Wilson R, Kris M, Varmus H (2004) EGF receptor gene mutations are common in lung cancers from “never smokers” and are associated with sensitivity of tumors to gefitinib and erlotinib. Proc Natl Acad Sci USA 101:13306-13311
10. Pao W, Miller V A, Politi K A, Riely G J, Somwar R, Zakowski M F, Kris M G, Varmus H (2005) Acquired resistance of lung adenocarcinomas to gefitinib or erlotinib is associated with a second mutation in the EGFR kinase domain. PLoS medicine 2:e73
11. Pawelek J M (2005) Tumour-cell fusion as a source of myeloid traits in cancer. The lancet oncology 6:988-993
12. Margolis R L (2005) Tetraploidy and tumor development. Cancer cell 8:353-354
13. Yuan H, Wang Z, Gao C, Chen W, Huang Q, Yee J K, Bhatia R, Chen W (2010) BCR-ABL gene expression is required for its mutations in a novel KCL-22 cell culture model for acquired resistance of chronic myelogenous leukemia. The Journal of biological chemistry 285:5085-5096
14. Akaike H (1974) A new look at the statistical model identification. IEEE Transactions on Automatic Control 19:716-723
15. Michor F, Hughes T P, Iwasa Y, Branford S, Shah N P, Sawyers C L, Nowak M A (2005) Dynamics of chronic myeloid leukaemia. Nature 435:1267-1270
16. Friedl P (2005) Cell fusion: new mechanisms of plasticity in cancer? The lancet oncology 6:916-918
17. Lu X, Kang Y (2009) Cell fusion as a hidden force in tumor progression. Cancer research 69:8536-8539
18. Peng B, Lloyd P, Schran H (2005) Clinical pharmacokinetics of imatinib. Clinical pharmacokinetics 44:879-894
19. Sharma S V, Bell D W, Settleman J, Haber D A (2007) Epidermal growth factor receptor mutations in lung cancer. Nature reviews 7:169-181
20. Ciardiello F, Tortora G (2008) EGFR antagonists in cancer treatment. N Engl J Med 358:1160-1174
21. Bell D W, Gore I, Okimoto R A, Godin-Heymann N, Sordella R, Mulloy R, Sharma S V, Brannigan B W, Mohapatra G, Settleman J, Haber D A (2005) Inherited susceptibility to lung cancer may be associated with the T790M drug resistance mutation in EGFR. Nature genetics 37:1315-1316

What is claimed is:

1. A method for identifying or classifying a gene mutation type associated with acquired drug resistance of cancer comprising:
determining a total copy number (N) of a susceptible gene in a cancer cell;
identifying a mutant copy number of the susceptible gene;
determining a mutant copy number sufficient to cause acquired drug resistance (M); and
comparing N with M to identify or classify the gene mutation type in the cancer cell.

2. The method of claim 1, wherein the mutation type is a single copy mutation type when N is 1 and M is 1 (M=N=1).

3. The method of claim 1, wherein the gene mutation type is a dominant mutation type when N is 2 or more and the number of point mutations sufficient to cause resistance is M=1.

4. The method of claim 1, wherein the gene mutation type is an intermediate mutation type when N is 3 or more and the mutant copy number sufficient to cause resistance is more than one, but less than the total copy number N (1<M<N).

5. The method of claim 1, wherein the gene mutation type is a recessive genetic drug resistance mechanism when N is 2 or more (N≧2) and the mutant copy number sufficient to cause resistance is equal to the number of gene copies (M=N≧2).

6. The method of claim 1, wherein the gene mutation type is a point mutation.

7. The method of claim 1, wherein the gene mutation type is associated with a cancer treatment selected from imatinib, erlotinib or gefitinib.

8. The method of claim 1, wherein determining the total copy number of the susceptible gene is accomplished by a gene sequencing method.

9. The method of claim 1, wherein determining the total copy number of the susceptible gene is accomplished by a gene amplification method.

10. The method of claim 1, wherein the classification of the genetic drug resistance profile indicates a preferred treatment regimen.

11. The method of claim 1, wherein the mutant copy number sufficient to cause resistance is determined by a computer system that calculates one or more equations associated with a mathematical or computational model.

12. A method for identifying a dominant mutation type associated with acquired drug resistance of cancers in a population of cancer cells comprising:
calculating a number of cells having 0, 1 or 2 mutant copies of a gene susceptible to acquired resistance at a series of predetermined time intervals;
generating a series of simulated growth kinetics graphs for cells having 0, 1 or 2 mutant copies of the susceptible gene at each time interval;
comparing the series of simulated growth kinetics graphs to experimentally determined growth kinetics data; and
determining the cancer cells acquire resistance to a drug through a dominant mutation type when the series of simulated growth kinetics graphs fit the experimentally determined growth kinetics data.

13. The method of claim 12, wherein the number of cells having 0, 1 or 2 mutant copies of the gene susceptible to acquired resistance is calculated based on computational models using an experimentally determined constant growth rate and an experimentally determined constant mutation rate.

14. The method of claim 13, wherein the experimentally determined constant growth rate is determined by counting cells on a hemocytometer at given time points after treatment with a drug associated with acquired resistance.

15. The method of claim 13, wherein the experimentally determined constant mutation rate is determined by a soft agar colony formation assay.

16. The method of claim 12, wherein the predetermined time interval is approximately 4 hours to one day.

17. The method of claim 12, wherein the experimental growth kinetics are determined by a cell viability assay after treatment with a drug associated with acquired resistance.

18. The method of claim 17, wherein the drug is imatinib, erlotinib or gefitinib.

19. The method of claim 12, wherein the mutant copy of the susceptible gene contains a point mutation.

20. A method for selecting, modifying, monitoring or predicting a response to a cancer treatment regimen for a cancer patient comprising:
identifying a cancer as having one or more mutations associated with acquired resistance to one or more cancer drugs;
determining a gene mutation type associated with acquired drug resistance of cancers for each of the one or more mutations as in claim 1; and
selecting, modifying or monitoring a cancer treatment regimen based on the mutation type.
